(19) United States
(12) Patent Application Publication
(10) Pub. No.: US 2026/0125461 A1
(43) Pub. Date: May 7, 2026

(54) Nanobody targeting IL-23A and application thereof

(71) Applicant: Beijing MEBIO Biotechnology Co., Ltd., Beijing (CN)

(72) Inventors: Fan ZHANG, Beijing (CN); Jian MA, Beijing (CN); Lin MEI, Beijing (CN); Jinxie ZHANG, Beijing (CN); Ran LUO, Beijing (CN)

(21) Appl. No.: 19/317,201

(22) Filed: Sep. 3, 2025

(30) Foreign Application Priority Data
Nov. 6, 2024 (CN) 202411575470.0

(51) Int. Cl. C07K 16/24

(52) U.S. Cl. CPC C07K 16/244; C07K 2317/565; C07K 2317/567; C07K 2317/569

(57) ABSTRACT

The present invention discloses a nanobody targeting IL-23A and an application thereof, and relates to the technical field of biological medicine. The nanobody VVH1 includes framework regions and complementary determining regions; the framework regions include VVH1FR-H1, VVH1FR-H2, VVH1FR-H3 and VVH1FR-H4, and the amino acid sequences of VVH1FR-H1, VVH1FR-H2, VVH1FR-H3 and VVH1FR-H4 are respectively shown as SEQ ID NOs. 4-7; and the complementary determining regions include VVH1CDR-H1, VVH1 CDR-H2 and VVH1 CDR-H3, and the amino acid sequences of VVH1 CDR-H1, VVH1 CDR-H2 and VVH1 CDR-H3 are respectively shown as SEQ ID NOs. 1-3. The nanobody has high binding activity with IL-23A, has the characteristics of small molecular weight, good stability, good tissue wettability, weak immunogenicity and the like compared with a traditional antibody, and can be applied to the preparation of an antibody drug targeting IL-23A so as to treat related diseases through combination with IL-23A.

### CROSS-REFERENCE TO SEQUENCE LISTING

The Sequence Listing XML file entitled “SequenceListing_XML.xml,” created on Aug. 8, 2025, and having a size of 11,399 bytes, is incorporated herein by reference in its entirety pursuant to 37 C.F.R. 1.821 (c) (1).

### TECHNICAL FIELD

The present invention relates to the technical field of biological medicine, and more particularly, to a nanobody targeting IL-23A and an application thereof.

### BACKGROUND ART

IL-23, as a member of the IL-12 cytokine family, is a heterodimer pro-inflammatory cytokine composed of two subunits of IL-23A (p19) and IL-12/23B (sharing p40 with IL-12) which are bound by a covalent disulfide bond. In 2003, it was found that the two subunits of IL-23 had different functions, wherein IL-23A was mainly produced by macrophages and dendritic cells, and was also proved to have a capability of coordinating the activity of immune cells, thereby playing a key role in the pathogenesis of immune inflammatory diseases.

A traditional antibody consists of two identical heavy-chain polypeptides and two identical light-chain polypeptides, wherein a heavy chain consists of one variable region and three constant regions, and a light chain consists of one variable region and one constant region. In 1993, Belgian scientist Hamers-Casterman and his team first reported a heavy-chain antibody found in camelid blood that naturally lacks light chains, which contains only one heavy-chain variable region (VHH) and two conventional constant regions. Single-domain antibodies each composed of only heavy-chain variable regions, also referred to as nanobodies (Nbs), may be obtained by cloning the variable region.

As a novel antibody, the nanobody has small size and molecular weight, strong tissue permeability, weak immunogenicity and other biological characteristics, and also has production characteristics of low production cost, high yield, capability of being produced and expressed in a variety of microbial systems in large quantities and no need for post-translational modification, as well as the capability of specifically identifying some hidden epitopes and other application advantages, thereby laying a good foundation for clinical development or industrial production.

Based on the above content, IL-23A may be used as a potential target for specific treatment of immune diseases, but the current clinical drug research on this target is still very limited, so the development of novel antibodies against this target is of great significance.

### SUMMARY OF THE INVENTION

An object of the present invention is to provide a nanobody targeting IL-23A and an application thereof, to solve the above problems existing in the prior art. The nanobody has high binding activity with IL-23A, has the characteristics of small molecular weight, good stability, good tissue wettability, weak immunogenicity and the like compared with a traditional antibody, and can be applied to the preparation of an antibody drug targeting IL-23A.

In order to fulfill the above object, the present invention provides the following technical solution:
the present invention provides a nanobody VVH1 targeting IL-23A, wherein the nanobody VVH1 includes framework regions and complementary determining regions; the framework regions include VVH1 FR-H1, VVH1 FR-H2, VVH1 FR-H3 and VVH1 FR-H4, and the amino acid sequences of VVH1 FR-H1, VVH1 FR-H2, VVH1 FR-H3 and VVH1 FR-H4 are respectively shown as SEQ ID NO. 4, SEQ ID NO. 5, SEQ ID NO. 6 and SEQ ID NO. 7; and the complementary determining regions include VVH1 CDR-H1, VVH1 CDR-H2 and VVH1 CDR-H3, and the amino acid sequences of VVH1 CDR-H1, VVH1 CDR-H2 and VVH1 CDR-H3 are respectively shown as SEQ ID NO. 1, SEQ ID NO. 2 and SEQ ID NO. 3.

The present invention further provides a coding gene, including the above-mentioned nanobody VVH1.

The present invention further provides a recombinant vector, including the above-mentioned coding gene.

The present invention further provides a transformed cell, including the above-mentioned recombinant vector.

The present invention further provides an application of the above-mentioned coding gene, recombinant vector or transformed cell in the preparation of the above-mentioned nanobody VVH1.

The present invention further provides an application of the above-mentioned nanobody VVH1 in the preparation of an antibody drug targeting IL-23A.

The present invention further provides an antibody drug targeting IL-23A, and an active ingredient includes the above-mentioned nanobody VVH1.

Further, the antibody drug also includes pharmaceutically acceptable accessories.

The present invention discloses the following beneficial effects.

According to the present invention, after a large number of studies, the nanobody VVH1 targeting IL-23A is obtained by screening. This nanobody has high binding activity with IL-23A, has the characteristics of small molecular weight, good stability, good tissue wettability, weak immunogenicity and the like compared with a traditional antibody, and can be applied to the preparation of an antibody drug targeting IL-23A so as to treat related diseases (e.g., inflammatory diseases or autoimmune diseases) through combination with IL-23A.

### BRIEF DESCRIPTION OF THE DRAWINGS

To describe the technical solutions in the examples of the present invention or in the prior art more clearly, the following briefly introduces the accompanying drawings required for describing the examples. Apparently, the accompanying drawings in the following description show merely some examples of the present invention, and a person of ordinary skill in the art may still derive other drawings from these accompanying drawings without paying creative efforts.

FIG. 1 is a sedimentation equilibrium experiment result in which a nanobody VHH1 binds to IL-23A;

FIG. 2 is a prediction analysis diagram in which a nanobody binds to an IL-23A epitope, wherein A is a schematic diagram in which a nanobody VHH1 binds to a truncated protein Epitope A; B is a schematic diagram in which the nanobody VHH1 binds to a truncated protein Epitope B; and

FIG. 3 is a schematic structural diagram of a composition where a nanobody VHH1 is docked with IL-23A, wherein yellow represents IL-23A, purple red represents an epitope region that predicts possible binding of the antibody, pure white represents the nanobody, and red, green and blue regions marked on the antibody correspond to CDR-H1, CDR-H2 and CDR-H3 regions of the antibody, respectively.

### DETAILED DESCRIPTION OF THE INVENTION

A variety of exemplary embodiments of the present invention are described in detail now. The detailed description should not be considered as a limitation of the present invention, but should be understood as a more detailed description of certain aspects, characteristics and embodiments of the present invention.

It should be understood that the terms described in the present invention are only intended to describe special embodiments and are not intended to limit the present invention. In addition, a numerical range in the present invention should be understood as further specifically disclosing each intermediate value between upper and lower limits of this range. The intermediate value in any stated value or stated range, and each smaller range between any other stated value or the intermediate value in said range are also included in the present invention. The upper and lower limits of these smaller ranges may be included in or excluded from the ranges independently.

Unless otherwise defined, all technical and scientific terms used herein have the same meaning as commonly understood by a person of ordinary skill in the art. Although the present invention only describes the preferred methods and materials, any method and material similar or equivalent to that described herein may also be used in the implementation or test in the present invention. All references in the present specification are incorporated by reference for the purpose of disclosing and describing methods and/or materials related to said literature. In the event of a conflict with any incorporated literature, it should be subject to the present specification.

Without departing from the scope or spirit of the present invention, a variety of improvements and changes may be made to the specific embodiments in the specification of the present invention, which is obvious to a person skilled in the art. Other embodiments obtained from the specification of the present invention are obvious to a person skilled in the art. The specification and examples of the present invention are illustrative only.

The terms used herein such as “including”, “comprising”, “having”, and “containing” are all open terms, which mean “include but not limited to”.

The description of the sequences involved in the present application is provided in Table 1.

TABLE 1
Sequence information.

| Description | Sequences |
|---|---|
| CDR-H1 general | LRLSCAASGX₁X₂X₃X₄X₅X₆X₇ |
| formula | |
| CDR-H2 general | IX₈X₉X₁₀GX₁₁-T |
| formula | |
| CDR-H3 general | X₁₂AX₁₃X₁₄X₁₅X₁₆X₁₇X₁₈X₁₉-- |
| formula | X₂₀X₂₁X₂₂X₂₃DY |
| VHH1 CDR-H1 | LRLSCATSAFIFRLNT |
| | (SEQ ID NO. 1) |
| VHH1 CDR-H2 | VTIDGST (SEQ ID NO. 2) |
| VHH1 CDR-H3 | KADRRIMMRTYDY |
| | (SEQ ID NO. 3) |
| VHH1 FR-H1 | EVQLVESGGGLVQAGGSLRLSCATS |
| | (SEQ ID NO. 4) |
| VHH1 FR-H2 | MGWYRQAPGKQRELVAM |
| | (SEQ ID NO. 5) |
| VHH1 FR-H3 | NYADSVKGRFTISRDSTKNTMYLQMNS |
| | LKPEDTAVYYC (SEQ ID NO. 6) |
| VHH1 FR-H4 | WGPGTQVTVSSEPKTPKPQP |
| | (SEQ ID NO. 7) |
| VHH1 amino | EVQLVESGGGLVQAGGSLRLSCATSA |
| acid | FIFRLNTMGWYRQAPGKQRELVAMVT |
| sequence | IDGSTNYADSVKGRFTISRDSTKNTM |
| | YLQMNSLKPEDTAVYYCKADRRIMMR |
| | TYDYWGPGTQVTVSSEPKTPKPQP |
| | (SEQ ID NO. 8) |

### EXAMPLE 1

### 1. Preparation of Nanobodies

According to the mutation treatment combined with general formulas of nanobodies, a plurality of nanobodies were designed and obtained by expression and purification. These nanobodies were composed of four framework regions (FRs) and three complementary determining regions (CDRs), referred to as FR-H1, CDR-H1, FR-H2, CDR-H2, FR-H3, CDR-H3 and FR-H4.

The nanobodies had the following general formula:
CDR-H1, having the following structure: LRLSCAASGX1X2X3X4X5X6X7;
CDR-H2, having the following structure: IX8X9X10GX11-T; and
CDR-H3, having the following structure: X12AX13X14X15X16X17X18X19-X20X21X22X23DY, wherein,
X₁ was selected from (i) amino acid residues F, R, S and (ii) amino acid residues conservatively substituted relative to (i);
X₂ was selected from (i) amino acid residues I, T and (ii) amino acid residues conservatively substituted relative to (i);
X₃ was selected from (i) amino acid residues F, Y, L and (ii) amino acid residues conservatively substituted relative to (i);
X₄ was selected from (i) amino acid residues T, R, S and (ii) amino acid residues conservatively substituted relative to (i);
X₅ was selected from (i) amino acid residues L, T, G, F and (ii) amino acid residues conservatively substituted relative to (i);
X₆ was selected from (i) amino acid residues N, Y and (ii) amino acid residues conservatively substituted relative to (i);
X₇ was selected from (i) amino acid residues T, E, F and (ii) amino acid residues conservatively substituted relative to (i);
X₈ was selected from (i) amino acid residues T, N, S and (ii) amino acid residues conservatively substituted relative to (i);
X₉ was selected from (i) amino acid residues I, W, S and (ii) amino acid residues conservatively substituted relative to (i);
X₁₀ was selected from (i) amino acid residues D, R, S and (ii) amino acid residues conservatively substituted relative to (i);
X₁₁ was selected from (i) amino acid residues G, I, S and (ii) amino acid residues conservatively substituted relative to (i);
X₁₂ was selected from (i) amino acid residues K, A, N and (ii) amino acid residues conservatively substituted relative to (i);
X₁₃ was selected from (i) amino acid residues D, R, E and (ii) amino acid residues conservatively substituted relative to (i);
X₁₄ was selected from (i) amino acid residues R, G and (ii) amino acid residues conservatively substituted relative to (i);
X₁₅ was selected from (i) amino acid residues F, R, W and (ii) amino acid residues conservatively substituted relative to (i);
X₁₆ was selected from (i) amino acid residues I, G, E and (ii) amino acid residues conservatively substituted relative to (i);
X₁₇ was selected from (i) amino acid residues M, I, S and (ii) amino acid residues conservatively substituted relative to (i);
X₁₈ was selected from (i) amino acid residues L, R and (ii) amino acid residues conservatively substituted relative to (i);
X₁₉ was selected from (i) amino acid residues T, I and (ii) amino acid residues conservatively substituted relative to (i);
X₂₀ was selected from (i) amino acid residues M, P, T and (ii) amino acid residues conservatively substituted relative to (i);
X₂₁ was selected from (i) amino acid residues R, A, N and (ii) amino acid residues conservatively substituted relative to (i);
X₂₂ was selected from (i) amino acid residues T, L, Q, R and (ii) amino acid residues conservatively substituted relative to (i); and
X₂₃ was selected from (i) amino acid residues Y, P, Q and (ii) amino acid residues conservatively substituted relative to (i).

### 2. Screening of Specifically Bound Nanobodies

By ELISA detection of the reactogenicity of a purified nanobody and an IL-23A important functional protein, the nanobodies whose ELISA results were positive and OD values were relatively high were picked out. Bacteria liquid PCR was carried out for positive clones, nanobody plasmids with positive PCR results were extracted for sequencing, and the sequencing results were shown in Table 1. After the sequencing results were analyzed by DNAMAN, an amino acid sequence of a nanobody that can undergo a protein-specific reaction with IL-23A was obtained, named as VVH1. The amino acid sequence was shown in Table 1.

### 3. Calculation of Binding Free Energy

In order to determine the binding free energy between the nanobody VHH1 and IL-23A, an MM-PBSA method was used for calculation. The binding affinity between the nanobody VHH1 and IL-23A was calculated. Molecular docking could predict a main binding mode between the antibody and IL-23A at an atomic level, and the interactions between the antibody and IL-23A typically include hydrogen bonding, salt bridging, and T-T stacking, etc. The binding energy of the nanobody VVH1 prepared by the present invention was-89.47866 kcal/mol, which could prove that the antibody and an antigen are bound stably.

### 4. Sedimentation Equilibrium Experiment

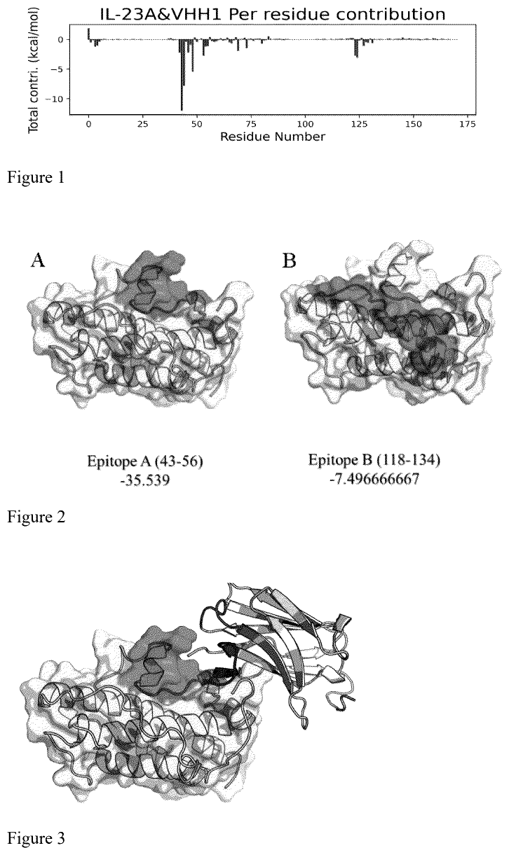

The sedimentation equilibrium experiment showed that the molecular weights of analogues were close to a calculated value of a cross-linked double-stranded monomer (FIG. 1), confirming that there was no oligomerization.

### 5. Determination of Binding Site

In order to determine a binding site of the nanobody and IL-23A, by taking the existing IL-23A plasmid in a laboratory as a template, an E. coli prokaryotic expression system was used to express an IL-23A truncated protein. Firstly, IL-23A was averagely truncated into two segments from the middle. Primers were designed to construct a recombinant plasmid for prokaryotic expression of two segments of truncated proteins (Epitope A and Epitope B). Then, they were respectively subjected to Western blotting reactions with the nanobody VVH1 to determine the binding forces of the nanobody VVH1 to different epitopes of IL-23A. The results were shown in FIG. 2.

### 6. Overall Spatial Structure of Nanobody and IL-23A and Spatial Distribution of Key Sites

In order to more intuitively show identified epitopes in the spatial structure, AlphaFold2 was used for homology modeling of the nanobody and IL-23A, and the data after modeling was processed and analyzed, showing the overall spatial structure of the nanobody VVH1 and IL-23A and the spatial distribution of key sites. The results were shown in FIG. 3.

The above examples are only a description of the preferred embodiments of the present invention, rather than limiting the scope of the present invention. Without departing from the design spirit of the present invention, a person of ordinary skill in the art may make various deformations and improvements on the technical solutions of the present invention, which should fall within the protection scope determined by the claims of the present invention.

1. A nanobody VVH1 targeting IL-23A, wherein the nanobody VVH1 comprises framework regions and complementary determining regions; the framework regions comprise VVH1 FR-H1, VVH1 FR-H2, VVH1 FR-H3 and VVH1 FR-H4, and the amino acid sequences of VVH1 FR-H1, VVH1 FR-H2, VVH1 FR-H3 and VVH1 FR-H4 are respectively shown as SEQ ID NO. 4, SEQ ID NO. 5, SEQ ID NO. 6 and SEQ ID NO. 7; and the complementary determining regions comprise VVH1 CDR-H1, VVH1 CDR-H2 and VVH1 CDR-H3, and the amino acid sequences of VVH1 CDR-H1, VVH1 CDR-H2 and VVH1 CDR-H3 are respectively shown as SEQ ID NO. 1, SEQ ID NO. 2 and SEQ ID NO. 3.

2. A coding gene, comprising the nanobody VVH1 according to claim 1.

3. A recombinant vector, comprising the coding gene according to claim 2.

4. A transformed cell, comprising the recombinant vector according to claim 3.

5. (canceled)


SEQUENCE LISTING (8 sequences)

SEQ ID NO: 1 - protein, 16 aa, synthetic construct
  REGION 1..16: note = VHH1 CDR-H1
LRLSCATSAFIFRLNT

SEQ ID NO: 2 - protein, 7 aa, synthetic construct
  REGION 1..7: note = VHH1 CDR-H2
VTIDGST

SEQ ID NO: 3 - protein, 13 aa, synthetic construct
  REGION 1..13: note = VHH1 CDR-H3
KADRRIMMRTYDY

SEQ ID NO: 4 - protein, 25 aa, synthetic construct
  REGION 1..25: note = VHH1 FR-H1
EVQLVESGGGLVQAGGSLRLSCATS

SEQ ID NO: 5 - protein, 17 aa, synthetic construct
  REGION 1..17: note = VHH1 FR-H2
MGWYRQAPGKQRELVAM

SEQ ID NO: 6 - protein, 38 aa, synthetic construct
  REGION 1..38: note = VHH1 FR-H3
NYADSVKGRFTISRDSTKNTMYLQMNSLKPEDTAVYYC

SEQ ID NO: 7 - protein, 20 aa, synthetic construct
  REGION 1..20: note = VHH1 FR-H4
WGPGTQVTVSSEPKTPKPQP

SEQ ID NO: 8 - protein, 128 aa, synthetic construct
  REGION 1..128: note = VHH1 amino acid sequence
EVQLVESGGGLVQAGGSLRLSCATSAFIFRLNTMGWYRQAPGKQRELVAMVTIDGSTNYADSVKGRFTISRDSTKNTMYLQMNSLKPEDTAVYYCKADRRIMMRTYDYWGPGTQVTVSSEPKTPKPQP